(19) United States
(12) Patent Application Publication
(10) Pub. No.: US 2024/0156601 A1
(43) Pub. Date: May 16, 2024

(54) Optimized Implant System

(71) Applicant: Karl Leibinger Medizintechnik GmbH & Co. KG, Mühlheim/Donau (DE)

(72) Inventors: Adem Aksu, VS-Schwenningen (DE); Frank Reinauer, Emmingen-Liptingen (DE); Tobias Wolfram, Dreieich (DE)

(21) Appl. No.: 18/549,734

(22) PCT Filed: Jan. 24, 2022
(86) PCT No.: PCT/EP2022/051440

§ 371 Date: Sep. 8, 2023

(30) Foreign Application Priority Data
Mar. 11, 2021 (DE) 10 2021 202 393.2

(51) Int. Cl. A61F 2/28; A61F 2/30; A61N 5/10

(52) U.S. Cl. CPC A61F 2/2803; A61F 2/2875; A61N 5/10; A61F 2002/30056

(57) ABSTRACT

In order to improve local specificity and adjustability of the radiant power in radiation diagnostics and in post-operative radiation therapy, the invention devises an implant system (100, 100′, 100″, 100′″) having a structural part (50, 50′, 50″, 50′″) that can be implanted in a damaged tissue region (30) and has at least a first region (10a, 10b, 10c, 10d) made of a first material and at least a second region (20a, 20b, 20c, 20d) made of a second material, the first region (10a, 10b, 10c, 10d) being designed to support the structural part (50, 50′, 50″, 50′″) and being substantially impermeable to a predetermined radiation for the purpose of diagnostics or for medical radiation therapy, and the second region (20a, 20b, 20c, 20d) being designed to complement the first region (10a, 10b, 10c, 10d) to the structural part (50, 50′, 50″, 50′″) and further comprising at least one portion (22a, 2b, 22c, 22d) that is structurally modified. The portion (22a, 2b, 22c, 22d) of the second region (20a, 20b, 20c, 20d) is permeable to the predetermined radiation for the purpose of diagnostics or for medical radiation therapy.

The present invention relates to an implant system comprising a structural part which is implantable into a damaged tissue area and which has at least one first region formed from a first material and at least one second region formed from a second material, wherein the first and the second material can be identical or different.

In the case of correction of bone-side defects which are caused by tumors for example and which are surgically replaced by implants, it is known to use biocompatible materials in which appropriate metals and their alloys are used. Such implants are known, for example, from DE 10 2005 003 188 A1.

In radiation therapy, the cancer cells are destroyed by means of ionizing radiation or particle radiation. The radiation damages the genetic material of the cells, and so cell division stops and the cells perish. As a result, the tumors become smaller or disappear. For example, in percutaneous radiation therapy, irradiation is carried out from a distance from outside the body. In this case, very high-energy radiation is generated, for example, by means of linear accelerators, which direct photon beams (here: hard X-radiation) onto the tumor and destroy the cancer cells.

However, with respect to its specificity as regards location and power, radiation therapy can be adversely affected by the shadowing or absorption effect of implant systems used, this also applying to radiodiagnostic applications, and it is therefore often not usable in a defined and controlled manner when they are present. Accordingly, the result obtained in radiation therapy is deficient or inadequate. In many cases, however, there is no other choice here but to accept the adverse effects of the deficits described as well as the associated consequential problems. Alternatively, the necessary reconstruction may otherwise be carried out only temporarily, minimalistically or not at all, which in turn can lead to other problems. Generally, the necessary surgery that should be done can only be done after completion of radiation therapy.

It is therefore an object of the present invention to provide an implant system which allows an improvement in localization and in setting radiant power both in radiation diagnostics and in the use of postoperative radiation therapy.

The object is achieved by an implant system having the features of claim 1. In particular, the solution according to the invention consists in, in the case of an implant system of the type mentioned at the start, the first region being arranged to support the structural part and being substantially impenetrable for a specified radioactive radiation for medical radiation therapy. At the same time, the second region is arranged to supplement the first region to form the structural part and is provided with at least one section having a structural modification, wherein the section of the second region is penetrable for the specified radioactive radiation for medical radiation therapy.

The implant system according to the invention can thus achieve an optimized input of radiation by providing on the structural part a first region composed of a first material and a second region composed of a second material, of which the first region forms, for example, the mechanical load-bearing component, whereas the second region is in the form of an implant component, for example consisting of a raw material which supports radiation therapy. This allows improved location accuracy and power adjustment for radiation therapy care or radiodiagnostic care that is improved in a patient-specific manner.

Preferred developments of the implant system according to the invention can be found in the relevant dependent claims.

In an advantageous development of the prosthetic part, the penetrability of the second region of the structural part can be suitably achieved by the second material being different from the first material. In this way, the functional assignment of the particular region can be achieved by making the optimal choice of material in conjunction with the other region.

A further stable and easy-to-handle development can be designed in such a way that the first region completely envelops the second region and can thereby stabilize and protect it, and so in this development of the implant system the second region is in the form of an insert in the first region.

A further preferred development can then in particular consist in the at least structurally modified section having a lower material density than its surrounding area. It is also conceivable to provide multiple structurally modified sections having the same or, if necessary, different material thicknesses.

Particularly accurate irradiation of the tumor, in which the healthy tissue in the surrounding area can be better protected, is achieved by means of another development in which the second region is in the form of a cut-out in the first region, and so the second region ensures a completely unimpeded passage of radiation through the structural part and input thereof into the patient-specific tissue area.

In another advantageous development, the second region can have a regular or irregular 3-dimensional scaffold structure, and so an optimized input of radiation is similarly achieved as in the case of a cut-out, but in this case the stability of the structural part is improved and a relatively large section is coverable with only a second region. Also conceivable are multiple second regions having a perforated grid structure on the structural part of the implant system according to the invention.

A satisfactory supporting effect and rigidity is experienced by the structural part in a preferred development in which the first material is a mechanically stable material. It can thus comprise metals, alloys thereof, polymers or combinations thereof. Particularly preferably, the metallic material of the first region can be titanium, titanium alloys, molybdenum, molybdenum alloys, magnesium, magnesium alloys, implantable stainless steel or a combination of at least two of these materials.

An advantageous supplementation of the first region to form the structural part, which an optimized input of radiation into tissue arranged behind it in relation to the radiation source, can be achieved by means of a development of the implant system according to the invention when the second material is in the form of a plastics material or a ceramic material or at least one resorbable component. Also conceivable here are combinations of materials.

Particularly preferably, the plastics material can be formed from at least one polymer, such as PEEK, PEKK, PE, PPSU or a combination of at least two of these materials. Equally preferred is a development in which the ceramic material is formed from aluminum oxide or zirconium oxide. Equally preferably, without being limited thereto, the resorbable component can be composed of, for example, HA, β-TCP, a combination of HA/β-TCP, β-TCP/Mg, PDLLA/Mg, PDLLA/P-TCP, PDLLA/CACC or similar materials.

Preference is given to using particle radiation, in particular electron radiation, or wave radiation, in particular X-radiation, as the intended radioactive radiation capable of destroying cancer cells by means of radiation. Owing to the design in multiple regions of differing transmissibility or penetrability for the intended radioactive radiation, said radiation can be applied in a particularly accurate and localized manner to a region at the power specifically required.

In an advantageous development of the implant system, the structural part can be arranged to replace, protect and/or cover a damaged bone area in the region of the cranium or mandible. The implant system is preferably precisely adaptable to patient-specific requirements and can comprise a multiplicity of the particular regions required.

The above configurations and developments can be combined with one other as desired, if appropriate. Further possible configurations, developments and implementations of the invention also encompass combinations of features of the invention that have not been explicitly mentioned, which features have been described above or will be described below with regard to the exemplary embodiments.

The invention will be more particularly elucidated below on the basis of exemplary embodiments in the figures of the drawing. Shown here in a partially schematized illustration are:

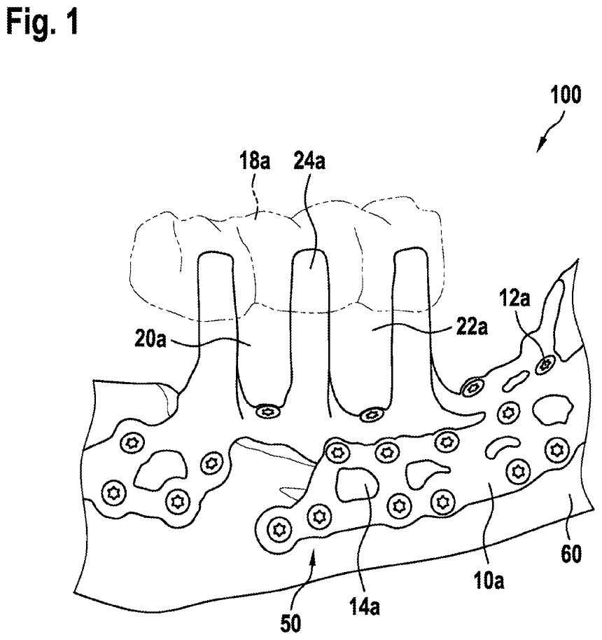

FIG. 1 a perspective side view of a first embodiment, in accordance with the invention, of the implantable prosthetic part in the region of a mandible comprising a structural part having a first and a second region and a structurally modified section on the second region;

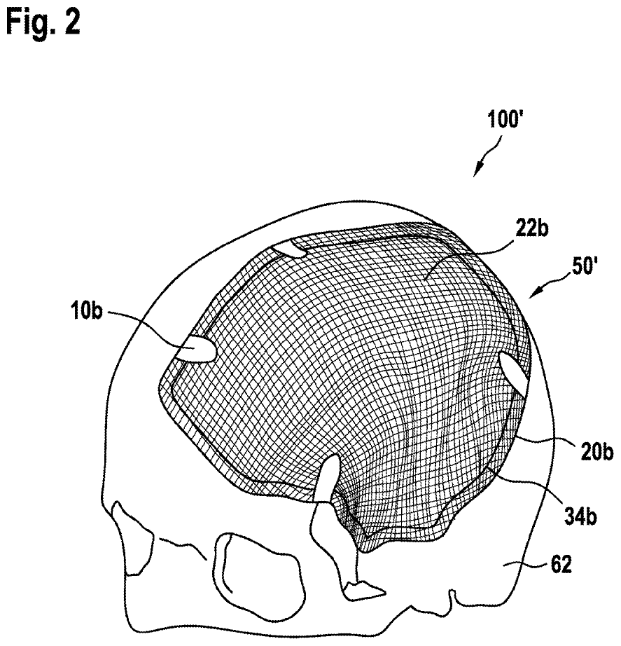

FIG. 2 a perspective side view of a second embodiment of the implantable prosthetic part in the region of a cranium comprising a structural part having a first and a second region and a structurally modified section on the second region;

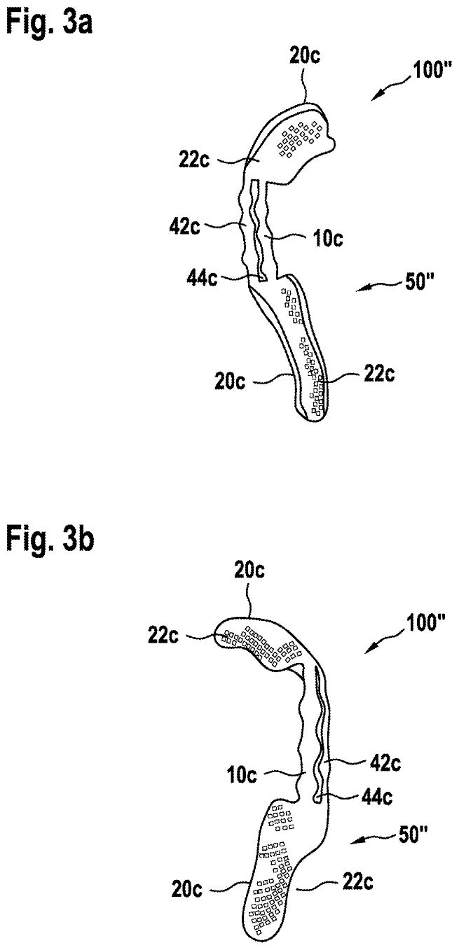

FIG. 3a, 3b perspective side views of a third embodiment of the implantable prosthetic part for disposition in the region of a mandible comprising a structural part having a first region and multiple second regions and structurally modified sections on the second regions; and

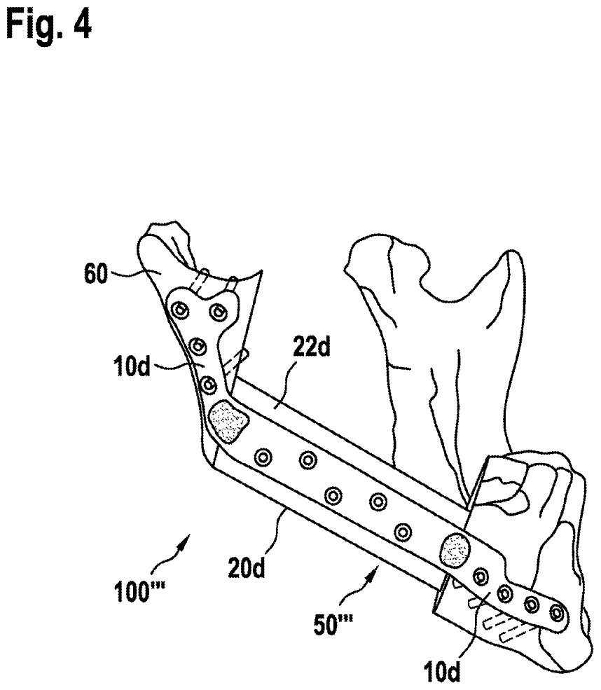

FIG. 4 a perspective side view of a fourth embodiment of the implantable prosthetic part in the region of the mandible comprising a structural part having a first and a second region and a structurally modified section.

In all the figures, identical or functionally identical elements and devices have been provided with the same reference signs, unless otherwise stated.

FIG. 1 shows a perspective side view of a first embodiment, in accordance with the invention, of the implantable prosthetic part, identified as a whole by 100, in the region of the mandible 60 comprising a structural part 50 having a first region 10a and a second region 20a and a structurally modified section 22a disposed in the second region.

In the illustration of FIG. 1, the first region 10a of the structural part 50 is composed of a titanium material in the form of a clamp which is fitted to the contour of the mandible and extends along it from a chin region depicted in the left-hand section of the illustration up to an ascending branch (ramus) of the mandible 60 in the right-hand section of the illustration. Here, the first region 10a of the structural part 50 is immobilized on the mandible 60 with fastening screws 12a.

Furthermore, provided on the first region 10a are cut-outs 14a which surrounds projections on the jaw bone and thus additionally prevent lateral displacement of the structural part and keep it stationary. Owing to their application on bone areas, the cut-outs 14a in question are suitable for passage of radioactive radiation, which is absorbed by the bone material disposed in the cut-outs 14a. In a middle section of the illustration of FIG. 1, three evenly spaced arms 24a, which are likewise composed of a titanium material, protrude from the first region 10a in the direction of the maxillary sinus and bear at their ends an artificial denture part 18a which closes the gap in the region of the teeth, said gap being present in the jaw as a result of resection of, for example, a tumor.

In relation to the mandible, the second region 20a is stretched out above it owing to the arms 24a and is in the form of a cut-out 26 in the first region 10a. The second region 20a formed by the arms 24a is arranged to supplement the first region 10a to form the structural part and has, by means of its solid structure which protrudes from the first region 10a and has a large clear dimension for the passage of radioactive radiation, a structural modification to provide substantially free passage through the relevant section of the mandible 60 that is traversed by a support structure which is formed by the arms 24a and which has sufficient stability to support or bear the denture part 18a and at the same time allow a sufficiently large input of radiation into the tissue to be irradiated present in the maxillary sinus.

FIG. 2 shows a perspective side view of a second embodiment of the implantable prosthetic part 100′ in the region of a cranium 62 comprising a structural part 50′ having a first and a second region 10b, 20b and a structurally modified section 22b on the second region 20b. In this second embodiment, the first region 10b of the structural part 50′ is formed by support parts which are irregularly disposed at the edge of an opening 34b created by surgical opening (trepanation) of the calvaria (decompressive craniectomy) and which protrude into the opening 34b substantially transversely to the edge. The support parts are composed of a titanium material. In contrast, the second region 20b here is formed by a regular perforated grid structure which covers the opening 34b and is held by the first region 10b. At the same time, the perforated grid structure of the second region 20b likewise composed of a titanium material forms a section 22b having a structural modification, supplements the first region 10b to form the structural part 50′ and is penetrable for the specified radioactive radiation.

FIGS. 3a and 3b show two perspective side views of a third embodiment of the implantable prosthetic part 100″ for disposition in the region of a mandible 60, not shown in FIGS. 3a and 3b, comprising in each case a structural part 50″ having a first region and multiple second regions 10c, 20c and structurally modified sections 22c on the second regions 20c. In the side views of FIGS. 3a and 3b, the structural parts 50′ are rotated approximately by 180° about their longitudinal axis. The structural part 50″ of FIGS. 3a and 3b has, in a middle section, a first region formed by two parallel webs 42c, the uniform distance of which forms a narrow gap 44c. Arranged at the ends of the two webs 42c is, in each case, a second region 22c of the structural part 50″, which in the use position grasps, in each case, a region of the mandible (not shown) and which, like the first region 10c, is composed of a metallic magnesium material.

The upper second region 20c of the structural part 50″ in the illustrations of FIGS. 3a and 3b is intended for grasping a chin section from the front, whereas the lower second region 20c in the illustrations of FIGS. 3a and 3b is intended for grasping the mandible 60 in the lateral region from the bottom. The two second regions 20c have a structural modification 22c and have an irregular perforated grid structure which is penetrated by the specified radioactive radiation for radiation therapy. The position of the perforated grid structure is specifically tailored to the lack of bone material and thus to the possibilities of radiation being able to be input into the tissue there as desired.

FIG. 4 shows a perspective side view of a fourth embodiment of the implantable prosthetic part 100′″ in the region of the mandible 60 comprising a structural part 50′″ having a first and a second region 10d, 20d and a structurally modified section 22d. In this case, the first region 10d is composed of a titanium material and disposed on a lateral region of the mandible 60, along which it extends and which it completely replaces in some parts of the course thereof. In a middle section of the first region 10d, a second region 20d of the structural part 50′″ is formed on the first region 10d as an insert having an elongated rhomboid cross-section, i.e., quadrangular cross-section, wherein the second region 20d is composed of a plastics material, more precisely a polymer, such as PEEK in this case. Thus, the second region 20d supplements the first part 10d to form the structural part 50′″ and has, by means of its section 22d, a structural modification such that the specified radioactive radiation, X-radiation here, penetrates the second region 20 and can be input into the soft tissue of the oral cavity that is situated behind it and is exposed by the removed bone.

The invention described above on the basis of preferred embodiments according to FIG. 1 to FIG. 4 therefore provides an implant system 100, 100′, 100″, 100′″ which allows an improvement in localization and in setting radiant power in the use of postoperative radiation therapy. For this purpose, in the case of the implantable prosthetic part 100, 100′, 100″, 100′″, the first region 10a, 10b, 10c, 10d of the structural part 50, 50′, 50″, 50′″ is arranged to support the structural part 50, 50′, 50″, 50′″ and to be substantially impenetrable for a specified radioactive radiation for medical radiation therapy, whereas the second region 20a, 20b, 20c, 20d is arranged to supplement the first region 10a, 10b, 10c, 10d to form the structural part 50, 50′, 50″, 50′″ and is provided with at least one section 22a, 2b, 22c, 22d having a structural modification. The section 22a, 2b, 22c, 22d of the second region 20a, 20b, 20c, 20d is penetrable by the specified radioactive radiation for medical radiation therapy.

Although the present invention has been described above on the basis of preferred exemplary embodiments, it is not restricted thereto, but is modifiable in a variety of ways. In particular, the invention can be altered or modified in many ways without departing from the essence of the invention.

1. An implant system comprising:
a structural part which is implantable into a damaged tissue area and which has at least one first region formed from a first material and at least one second region formed from a second material, wherein
the first region is arranged to support the structural part and is substantially impenetrable for a specified radiation for diagnostics or for medical radiation therapy;
the second region is arranged to supplement the first region to form the structural part and is provided with at least one section having a structural modification, and
the section of the second region is penetrable for the specified radiation for diagnostics or for medical radiation therapy.

2. The implant system as claimed in claim 1, wherein the second material is different from the first material.

3. The implant system as claimed in claim 1, wherein the second region is in the form of an insert in the first region.

4. The implant system as claimed in claim 1, wherein the second region is in the form of a cut-out in the first region.

5. The implant system as claimed in claim 1, wherein the at least structurally modified section has a lower material density than its surrounding area.

6. The implant system as claimed in claim 1, wherein the second region has a regular or irregular perforated grid structure.

7. The implant system as claimed in claim 1, wherein the first material is a mechanically stable material, in particular a metallic material.

8. The implant system as claimed in claim 7, wherein the metallic material is formed from titanium, titanium alloys, molybdenum, molybdenum alloys, magnesium, magnesium alloys, implantable stainless steel or a combination of at least two of these materials.

9. The implant system as claimed in claim 1, wherein the second material is in the form of a plastics material, ceramic material, composite or a resorbable combination of these.

10. The implant system as claimed in claim 9, wherein the plastics material is formed from at least one polymer such as PEEK (polyetheretherketone), PEKK (polyetherketoneketone), PE (polyethylene), PPSU (polyphenylsulfone) or a combination of at least two of these materials.

11. The implant system as claimed in claim 9, wherein the ceramic material is formed from aluminum oxide or zirconium oxide.

12. The implant system as claimed in claim 9, wherein the resorbable component is composed of HA (hydroxyapatite), β-TCP (beta-tricalcium phosphate), combinations of HA/β-TCP, β-TCP/Mg, PDLLA/Mg (poly-d,l-lactic acid), PDLLA/β-TCP, PDLLA/CaCC or similar materials.

13. The implant system as claimed in claim 1, wherein the radioactive radiation is particle radiation, in particular electron radiation, or wave radiation, in particular X-radiation.

14. The implant system as claimed in claim 1, wherein the structural part is designed to replace a damaged bone area in the region of the cranium or mandible.
